(19) United States
(12) Patent Application Publication
(10) Pub. No.: US 2020/0234829 A1
(43) Pub. Date: Jul. 23, 2020

(54) SYSTEMS AND METHODS FOR FACILITATING RESPONSE PREDICTION FOR A CONDITION

(71) Applicant: KONINKLIJKE PHILIPS N.V., EINDHOVEN (NL)

(72) Inventors: Ronald Leo Christiaan KOYMANS, Eindhoven (NL); Frank WARTENA, Eindhoven (NL); Lisa RUNCI, Eindhoven (NL)

(21) Appl. No.: 16/838,533

(22) Filed: Apr. 2, 2020

Related U.S. Application Data
(63) Continuation-in-part of application No. 14/488,437, filed on Sep. 17, 2014.

(51) Int. Cl. G16H 50/20; G16H 40/67; G16H 50/70; G16H 10/20; G06N 3/08; G06N 3/04

(52) U.S. Cl. CPC G16H 50/20; G16H 40/67; G06N 3/0454; G16H 10/20; G06N 3/084; G16H 50/70

(57) ABSTRACT

In certain embodiments, response prediction for a condition may be facilitated. In some embodiments, physiological data indicating a condition of the user may be obtained. A plurality of users related to the user may be determined based on the physiological data. Combined response data related to the users may be obtained, where the combined response data indicates actual responses of the users to presentation of predicted responses for conditions related to the condition of the user. Combined physiological data (indicating the related conditions) may be provided to a neural network for the neural network to predict responses for the related conditions. The combined response data may be provided to the second network as reference feedback to cause the neural network to configure its layers based the combined response data and the predicted responses.

### RELATED APPLICATION(S)

This application is a continuation-in-part of U.S. patent application Ser. No. 14/488,437, filed Sep. 17, 2014, which is hereby incorporated by reference herein in its entirety.

### FIELD OF THE INVENTION

The present invention relates to systems and methods for facilitating a response to a condition of a user, including, for example, response prediction for the condition of the user.

### BACKGROUND

When people can get access to a multitude of services, e.g. through a single portal or platform, it can be challenging to recommend services that are both relevant and suitable to them. Services in this context may be any kind of services that can be used by a user and that in any way may support the user or may be desired by the user.

### SUMMARY

Certain embodiments described herein relate to methods, apparatuses, and/or systems for facilitating a response to a condition of a user.

In some embodiments, data related to the user (e.g., data indicating a condition of the user or other data) may be obtained. A plurality of users may be determined as related to the user based on the user-related data. Based on the determination of the plurality of users, aggregated physiological data and aggregated response data related to the plurality of users may be obtained. As an example, the aggregated physiological data may indicate conditions of the plurality of users that relate to the condition of the user, and the aggregated response data may include data indicating actual responses of the users to presentation of predicted responses for conditions related to the condition of the user. Based on the aggregated physiological data and the aggregated response data, a response for the condition of the user may be generated and presented to the user.

In some embodiments, physiological data (e.g., data indicating the condition of the user) may be obtained. The plurality of users may be determined as related to the user based on the physiological data. Aggregated physiological data related to the plurality of users (e.g., data indicating the related conditions of the plurality of users) may be provided to a neural network for the neural network to predict responses for the related conditions. The aggregated response data related to the plurality of users (e.g., data indicating the actual responses of the users) may be provided to the neural network as reference feedback to cause the neural network to configure one or more layers of the neural network based the aggregated response data and the predicted responses.

Various other aspects, features, and advantages of the invention will be apparent through the detailed description of the invention and the drawings attached hereto. It is also to be understood that both the foregoing general description and the following detailed description are examples and not restrictive of the scope of the invention.

### BRIEF DESCRIPTION OF THE DRAWINGS

FIG. 1A shows a system for facilitating a response to a condition of a user or other response for the user, according to one or more embodiments.

FIG. 1B shows a machine learning model and data for configuring the machine learning model, according to one or more embodiments.

FIGS. 2-9 shows various flow charts and schematic diagrams for facilitating a response for a user, according to various embodiments.

### DETAILED DESCRIPTION

In the following description, for the purposes of explanation, numerous specific examples are set forth in order to provide a thorough understanding of example embodiments. It will be appreciated, however, by those having skill in the art that embodiments may be practiced without these specific details or with an equivalent arrangement.

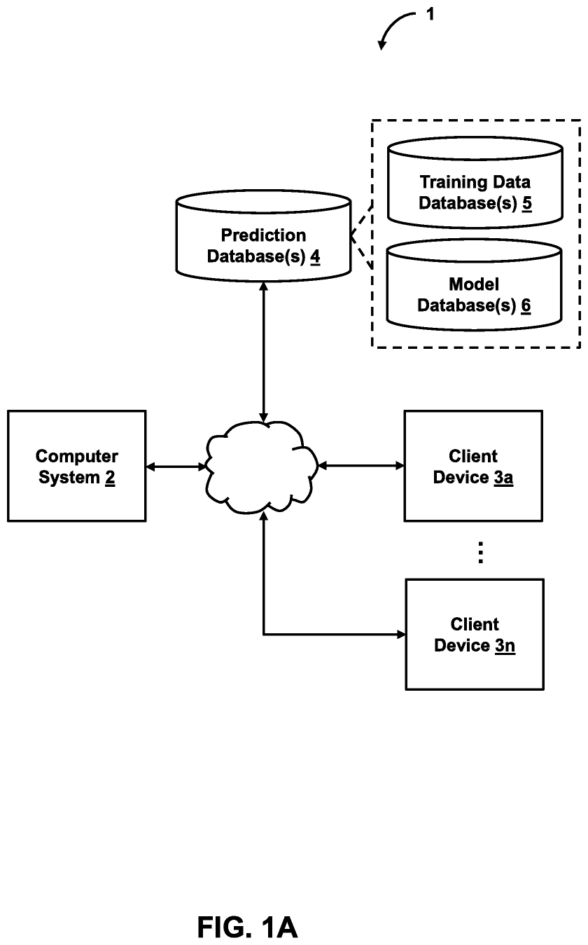

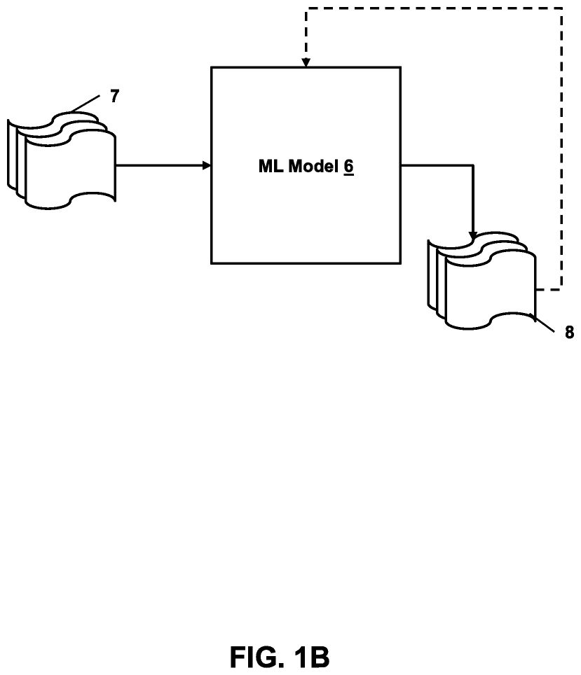

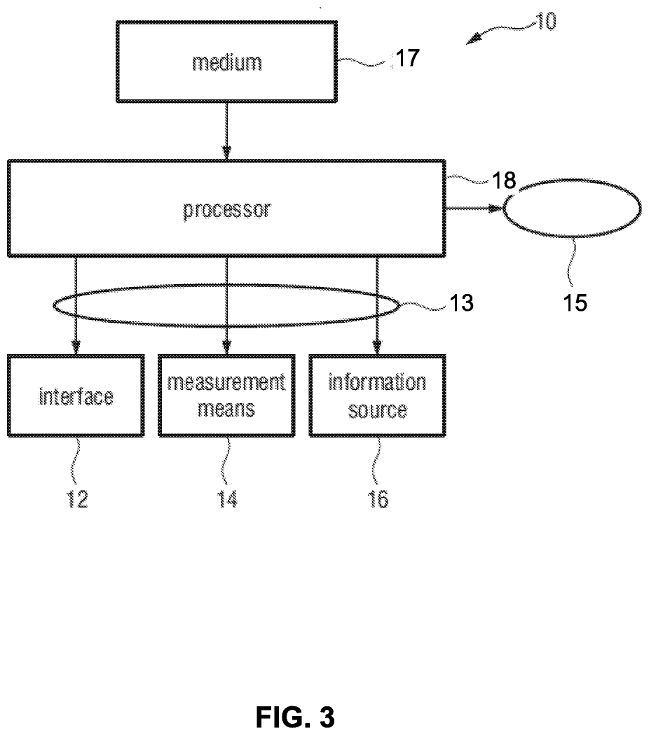

FIG. 1 shows a system 1 for facilitating prediction model training, in accordance with one or more embodiments. As shown in FIG. 1, system 1 may include computer system 2 (e.g., a server system or other computer system), client device 3 (or client devices 3a-3n), or other components. Computer system 2 may include one or more components of systems 10, 20, or 30 (see FIGS. 3, 5, 8) or other components. Each client device 3 may include any type of mobile terminal, fixed terminal, or other device. By way of example, client device 3 may include a desktop computer, a notebook computer, a tablet computer, a smartphone, a wearable device (e.g., smartwatch, spectacles, in-ear or over-ear device, headband, body-embedded device, etc.), or other client device. Users may, for instance, utilize one or more client devices 3 to interact with one another, one or more servers, or other components of system 1. It should be noted that, while one or more operations are described herein as being performed by particular components of computer system 2, those operations may, in some embodiments, be performed by other components of computer system 2 or other components of system 1. As an example, while one or more operations are described herein as being performed by components of computer system 2, those operations may, in some embodiments, be performed by components of client device 3. It should be noted that, although some embodiments are described herein with respect to machine learning models, other prediction models (e.g., statistical models or other analytics models) may be used in lieu of or in addition to machine learning models in other embodiments (e.g., a statistical model replacing a machine learning model and a non-statistical model replacing a non-machine-learning model in one or more embodiments).

In some embodiments, system 1 may utilize data related to the user to determine one or more other users as being related to the user. Based on aggregated data related to the other users, a response for one or more conditions of the user may be generated. In some embodiments, system 1 may obtain user profile data of the user, such as data indicating a condition, age, gender, nationality, geographical location, medications, social economics, or other aspect of the user. A plurality of users may be determined as being related to the user based on the user profile data. Based on the determination of the plurality of users, aggregated user profile data and aggregated response data related to the plurality of users may be obtained. As an example, the aggregated user profile data may indicate conditions of the plurality of users, ages, genders, nationalities, geographical locations, medications, social economics, or other aspects of the plurality of users, and the aggregated response data may include data indicating actual responses of the users (e.g., to presentation of predicted responses for conditions related to the condition of the user). Based on the aggregated user profile data and the aggregated response data, a response for the user may be generated.

In some embodiments, system 1 may provide the aggregated user profile data related to the plurality of users (e.g., data indicating the related conditions of the plurality of users) to a prediction model, and the prediction model may predict responses for the related conditions. The prediction model may be updated based on the predicted responses and the aggregated response data related to the plurality of users (e.g., data indicating the actual responses of the users). As an example, the aggregated response data related to the plurality of users may be provided to the prediction model as reference feedback, and the prediction model may update one or more portions of the prediction model based on the aggregated response data and the predicted responses. In this way, for example, the prediction model may be personalized for the user (e.g., personalized to the condition of the user or other parameters pertaining to the user) without first requiring a significant amount of the user's response data (e.g., data indicating the user's actual responses) or without any such user response data as the initial training set for the prediction model.

In some embodiments, the prediction model may include one or more neural networks or other machine learning models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it propagates to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

As an example, with respect to FIG. 1B, machine learning model 6 may take inputs 7 and provide outputs 8. In some embodiments, outputs 8 may be fed back to machine learning model 6 as input to train machine learning model 6 (e.g., alone or in conjunction with user indications of the accuracy of outputs 8, labels associated with the inputs, or with other reference feedback information). In some embodiments, machine learning model 6 may update its configurations (e.g., weights, biases, or other parameters) based on its assessment of its prediction (e.g., outputs 8) and reference feedback information (e.g., user indication of accuracy, reference labels, or other information). In some embodiments, where machine learning model 6 is a neural network, connection weights may be adjusted to reconcile differences between the neural network's prediction and the reference feedback. Some embodiments include one or more neurons (or nodes) of the neural network requiring that their respective errors are sent backward through the neural network to them to facilitate the update process (e.g., backpropagation of error). Updates to the connection weights may, for example, be reflective of the magnitude of error propagated backward after a forward pass has been completed. In this way, for example, the machine learning model 202 may be trained to generate better predictions.

In some embodiments, one or more prediction models may initially be trained or configured to be targeted for one or more sets of user classifications, such as a set of users that fall into one or more of an age group, gender category, nationality, geographical location or region, medication group (e.g., those are under taking certain medications), condition or condition type, social economic class, or other classifications. As an example, such prediction models may be stored in one or more database for later use (e.g., model database(s) 6 of prediction database(s) 4 or other databases). Based on a determination that a user falls under a particular set of user classifications, a prediction model initially trained or configured to be targeted for that set of user classifications may be selected, and an instance of the prediction model may obtained and further trained or configured based on the user profile data of the user to facilitate a response prediction for the user. As an example, the prediction model may initially be trained or configured for users in the 25-40 age range and earning $50,000-$85,000, and the prediction model may be selected for the user based on the user being within the foregoing age and income range. To further personalize the experience and accuracy for the user, an instance of the prediction model may be obtained and furthered trained or configured using data pertaining to the user (e.g., user profile and response data of the user) and aggregated data pertaining to users that are similar to the user (e.g., aggregated user profile data and response data of the similar users). In this way, for example, such prediction models may be specifically personalized for a given user, while reducing the amount of time and computational resources necessary to perform such personalization. As an example, because a prediction model is already pre-trained or configured for a larger set of users that already fall under one or more of the same classifications of the user, further personalization of the prediction model may be performed using a smaller training data set (e.g., smaller input training data, smaller reference feedback training data, etc.) than would otherwise have been required to achieve similar accuracy of predictions.

In some embodiments, prior to using training data to train a prediction model, the training data may be analyzed via a relevancy model to determine whether the prediction model would be influenced by one or more feature types represented by the training data. Some aspects (e.g., features) of the training data may have a greater influence on the prediction model's results as compared to other aspects. Depending on the results of the relevancy model, the training data may be updated to modify (e.g., suppress or boost) the amount of the influence these feature types have on the prediction model's results. In this way, the prediction model's results may be improved because the prediction model will be trained with optimized training data. For example, the training data used to train the prediction model may reduce or eliminate an impact of one or more particular feature types on the results of the prediction model. In this way, because of the reduction or elimination of such impact, the foregoing may improve the accuracy of the prediction model.

In some embodiments, system 1 may obtain one or more datasets for generating training data to be used to train the prediction model. In some embodiments, the datasets may be randomly selected from one or more data corpora. The datasets may include a plurality of feature types, and each dataset may include some or all of the plurality of feature types. In some embodiments, data including a set of different features may be provided as an input into a prediction model to obtain a result. The features may be of a same “type” or may be of different types. As an example, data including features, such as noise ratios, lengths of sound, relative power, etc., may serve as an input to a prediction model related to recognizing phonemes for speech recognition processes. As another example, data including features such as edges, objects, pixel information, may serve as an input to a prediction model related to computer vision analysis. As still yet another example, data including features, such as condition, age, gender, nationality, geographical location, medications, social economic information, may serve as an input to a prediction model related to financial applications. Each of the features (e.g., noise rations, lengths of sound, relative power, edges, objects, condition, age, gender, nationality, geographical location, medications, social economic information, or other features) may be different types of features. The feature type may relate to the genre of the prediction model (e.g., speech recognition models, computer vision models, etc.) or the different individual fields encompassed by a feature (e.g., length of sounds in units of time, income in units of dollars, etc.). As described herein, a feature type corresponds to a type of feature, i.e., what the feature represents. For example, the feature type of salary information corresponds to the feature salary, which may be used as an input to a financially-related prediction model.

Different feature types may influence an output of a given prediction model in different ways. An amount of influence a feature type has on the prediction model's output may vary. Depending on the amount of influence, a determination may be made prior to using the training data to train the prediction model as to whether the training data unknowingly causes the prediction model's results to favor or not favor a particular feature type or types. For instance, a determination should be made prior to training the prediction model as to whether the training data includes feature types having a threshold amount of influence on the prediction model's results as compared to other feature types. If so, a determination may also be made as to whether those feature types should influence the prediction model to their particular extent.

In some embodiments, prior to providing training data to the prediction model for training the prediction model, the training data may first be provided to a relevancy model. The relevancy model may determine how relevant one or more feature types are to the output of the prediction model. Alternatively, the training data may be provided to an irrelevancy model to determine how irrelevant one or more feature types are to the output of the prediction model. In some embodiments, a feature type determined to have a threshold amount of influence (or more) on the prediction model's results may indicate that the training data should be updated. In some embodiments, updating the training data may improve the accuracy and reliability of the prediction model because the amount of influence imparted by the feature type may be reduced. For example, the training data may be used to eliminate or reduce an impact of a feature type on the results of the prediction model. Therefore, if the relevancy model indicates that the datasets used to generate the training data include a feature type that has an amount of influence equal to or greater than the threshold amount of influence, then the training data may be updated. In this way, because of the reduction or elimination of such impact, the foregoing may improve the prediction model.

In some embodiments, as discussed herein, system 1 may determine one or more other users as being related to a user by determining that the other users are similar to the user based on the user's user profile data indicating characteristics of the user that are similar to characteristics of the other users (e.g., indicated by the other users' user profile data). As an example, two users may be determined to be similar based on one or more similarity criteria related to condition (e.g., a medical or other health condition), age, gender, nationality, geographical location, medications, social economics, etc. In one use case, for example, determining similarity of users may be based on profiling of users. On the basis of two profiles, a measure of their (relative) similarity can be defined that depends on the closeness of the parameters of the two profiles (e.g., age, gender).

In some embodiments, based on the determination the other users (e.g., the similar users), aggregated user profile data and aggregated response data related to the other users may be obtained. As an example, the aggregated user profile data may indicate conditions of the plurality of users, ages, genders, nationalities, geographical locations, medications, social economics, or other aspects of the plurality of users, and the aggregated response data may include data indicating actual responses of the users (e.g., to presentation of predicted responses for conditions related to the condition of the user). Based on the aggregated user profile data and the aggregated response data, a response for the user may be generated. In some embodiments, the predicted responses may include recommended use of one or more devices or services for the other users, and the actual responses of the other users may include actual usage of the devices or services by the other users (e.g., usage frequency, usage duration, or other usage data). In some embodiments, the generated response for the user may include recommended use of a device or service for the user.

In some embodiments, services used by similar users (in the past or present) are collected and evaluated. In some embodiments, this may further be understood as collecting various additional pieces of information regarding services, such as which kinds or types of services have been used, how long or to which extent services have been used, where services have been used, how often services have been used, etc.

In some embodiments, the systems and corresponding methods use the user profile and calculate to what extent the user profile matches the intended use of a service. An intended use of a service can include both information for which users this service is appropriate (e.g., a weight loss service is intended for users with a too high Body Mass Index and who are interested in losing weight) as well as information for which users this service is not appropriate (e.g., an extreme sports program is not intended for users with heart problems or osteoporosis).

In some embodiments, there are provided corresponding methods as well as a non-transitory computer-readable recording medium that stores therein a computer program product, which, when executed by a processor, causes the method disclosed herein to be performed.

In some embodiments, the instructions further cause the processor to determine the level of similarity for a plurality of users and to select similar users based on the determined level of similarity. This increases the accuracy and effectiveness of recommended services.

In some embodiments, the instructions further cause the processor to determine for collected services the level of satisfaction of similar users based on direct feedback from said similar users and/or actual usage of services by said similar users. For this purpose an interface for obtaining direct feedback and/or information about actual usage of services by users and/or measurement means for measuring actual usage of services by users including usage frequency and/or usage duration may be provided. Hence, the information for determining the level of similarity is preferably selected in an automated manner.

In some embodiments, the instructions further cause the processor to determine the level of similarity for a plurality of users based on the profile of the users, select similar users based on the determined level of similarity, determine the suitability of collected services describing their intended use with respect to a user's profile, and recommend a service for use by the particular user from the collected services based on the determined levels of satisfaction and the suitability of the collected services for the particular user. This further helps in the selection of recommendations that are most appropriate and effective for the particular user.

In some embodiments, the instructions further cause the processor to recommend a service for use by the particular user from the collected services based on the determined levels of satisfaction and based on the number of times a service was recommended to similar users and/or the number of similar users to which a service was recommended. This also increases the accuracy and effectiveness of recommended services.

In some embodiments, the instructions preferably further cause the processor to recommend a healthcare service for use by the particular user from the collected healthcare services based on the determined levels of satisfaction and based on the number of times a service was recommended to similar users and/or the number of similar users to which a service was recommended by a care provider. As mentioned above, the instructions further cause the processor to preferably take into account a reputation level of said care providers in the recommendation of a healthcare service.

In some embodiments, the instructions further cause the processor to determine for the respective collected services a combined satisfaction level from the determined level of satisfaction of similar users and to recommend a service for use by the particular user from the collected services based on the combined satisfaction level. The combined satisfaction level may e.g., be the average, a mean, a summation, etc. of the levels of satisfaction of similar users. This embodiment further increases the accuracy.

In some embodiments, the instructions further cause the processor to determine the suitability of a service for a user based on the profile of the user and the intended use of the service. Particularly, one or more exclusion criteria may be used for determining the suitability of a service for a user, an exclusion criterion indicating which users can or shall not use a particular service. Preferably, one or more exclusion criteria may be used that take into account availability, affordability and/or accessibility of a service for a user.

In some embodiments, input from recommendation givers may be used to determine if a service should be recommended for a particular user. In many cases a care provider will recommend specific services to their patients, as the user profile of these patients is known in the system. This prescribing behavior of care providers can be used to determine which services are endorsed by care providers for which type of users. By combining the prescribing behavior of care providers with a reputation measure of these care providers it is possible to improve the recommendations even further by giving care providers with a high reputation a stronger weight in the calculation of recommendations.

In some embodiments, the instructions further cause the processor to determine the reputation level of said recommendation givers, in particular of care providers, e.g., based on the number and/or quality of publications, the scientific H-index, recommendations, scores and/or ratings given by users, experts and/or other recommendation givers, and/or the outcome of prior use of services recommended by said recommendation giver, in particular of healthcare services recommended by a care provider.

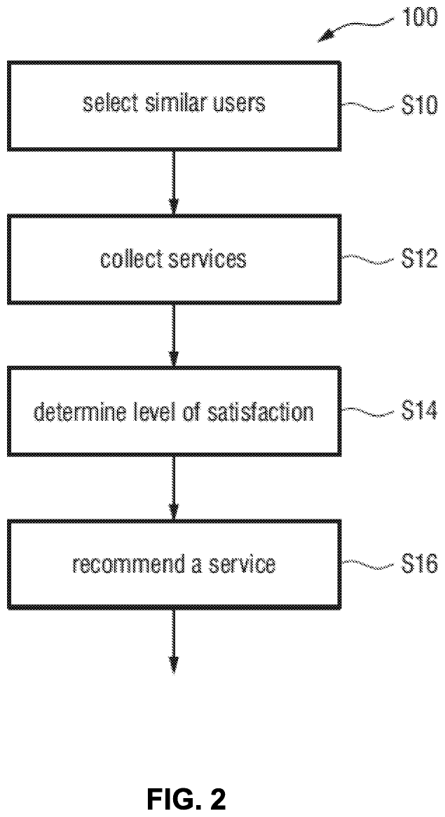

FIG. 2 shows a flow chart of a first embodiment of a method 100. In a first step S10 similar users that are similar to the particular user are selected from a plurality of users. In a second step S12 services used by similar users are selected. In a third step S14 for collected services the level of satisfaction of similar users using the respective collected service is determined. In a fourth step S16 a service is recommended for use by the particular user from the collected services based on the determined levels of satisfaction of similar users.

Similarity is e.g., based on user profiles for which a distance function is defined that computes (relative) closeness of profiles (closer means more similar), i.e. the (relative) similarity can be defined that depends on how (relatively) close the parameters of the two profiles (e.g., age, gender) are. A strongly simplified example, in which profiles only use age and gender as parameters, shall be used to explain this. Then the distance between user profiles could be defined as the difference in age plus 10 additional points in case the gender differs. So, the distance between a 30-year old female and a 40-year old female would be 10, but the distance between the same 30-year female and a 33-year old male would be 3+10=13 and therefore actually larger than the distance to the 40-year old female.

For determining (relative) similarity, a minimum threshold (minimal closeness) may be used. The group of similar users is restricted to have a minimum closeness to the particular user. In the example of the 30-year old female a minimum closeness of 12 would mean the 40-year old female would qualify but the 33-year old male would not.

As mentioned above, the problem of profile matching is generally known, and the known examples are much more specific, for instance a 35-years-old male, 1.82 cm tall, with strong interests in fantasy novels and Japanese comics, fair interest in politics and no interest in football.

The selection of services used by similar users generally depends on the application domain. One application domain is the healthcare domain. In that case services are generally prescribed to a user (patient) by a professional caregiver. In this setting the prescription of such services is well administered. In other domains this might be more difficult, but part of that knowledge could be in the public domain or otherwise potentially be acquired from the respective service providers. It should be noted that perfect knowledge about all the services used by similar users is generally not needed, the proposed idea still works with any (partial) knowledge being available or in situations in which there is some knowledge available about the use of certain services by other users.

The level of satisfaction may be based on various factors: user satisfaction surveys, service usage frequency and/or service usage duration. The first factor is explicitly related to satisfaction, the other two are implicitly related to satisfaction (where more frequent and longer users of a service are seen as more satisfied). Hence, the level of satisfaction of a user of a service can be determined in several ways. An often used method is direct feedback from a user, for instance by means of a questionnaire. Direct feedback from a user is complemented by taking the actual usage of the service by the user into account. The actual usage can give additional insights into important factors such as real service utilization (independent from candor or memory of the user) and relative scaling of service usefulness by different users. Actual usage of a service by a user through a portal or platform can be determined by IT tools such as monitoring and logging.

A corresponding first embodiment of a system 10 is depicted in FIG. 3. Said system comprises an interface 12 for obtaining direct feedback and/or information about actual usage of services by users. Such an interface may be any means by which a user can input feedback and/or information, e.g., a terminal station, a smartphone, a computer, a particular website that can be accessed by the user, etc. Generally, however, to obtain feedback by a user does not necessarily require a digital interface. The user can fill in a paper questionnaire that is later processed and transformed into computer-readable (e.g., digital) format automatically or manually.

Further, the system 10 comprises measurement means 14 for measuring actual usage of services by users including usage frequency and/or usage duration. Such measurement means 14 may be any means that are used by the user when using a service, e.g., a medical device that is used for applying a medical treatment, a computer that is used for reading a tutorial, a smartphone when getting instructions or reminders, etc. Further (not necessarily medical) devices that can track actual usage and may be used as measurement means 14 are all kinds of monitoring devices (including wearables) that form part of the service in question (e.g., a weight scale for a weight management service).

Further, an information source unit 16 may be provided for storing other input information 13 (e.g., information about services used by other users, user profiles, similarity data, etc.) for use by the processor 18 for generating recommendations 15. Said information storage unit 16 may e.g., be a storage medium 17 (e.g., on a workstation, a server, in a network, in the cloud, etc.), a user interface, a website or database that can be searched by the processor 18, etc.

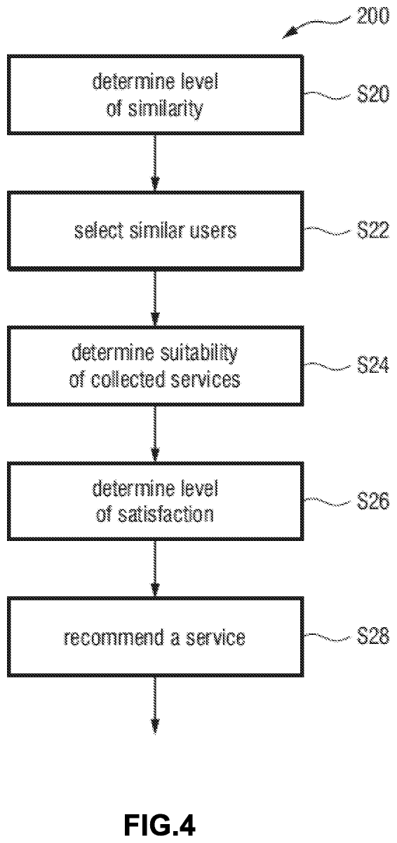

FIG. 4 shows a flow chart of a second embodiment of a method 200 which may be carried out by a system 1 depicted in FIG. 1. In a first step S20 the level of similarity for a plurality of users is determined based on the profile of the users. In a second step S22 similar users are selected from said plurality of users based on the determined level of similarity. In a third step S24 the suitability of collected services describing their intended use with respect to a user's profile is determined. In a fourth step S26 for collected services the level of satisfaction of similar users using the respective collected service is determined. In a fifth step S28, a service is recommended for use by the particular user from the collected services based on the determined levels of satisfaction and the suitability of the collected services for the particular user.

For instance, in an embodiment, one or more exclusion criteria for determining the suitability of a service for a user are used, wherein an exclusion criterion indicates which users can or shall not use a particular service. Such exclusion criteria may take into account availability, affordability and/or accessibility of a service for a user.

In another embodiment, the recommendation of a service may be made based on the determined levels of satisfaction and based on the number of times a service was recommended to similar users and/or the number of similar users to which a service was recommended.

Further, for the respective collected services a combined satisfaction level (e.g., an average, a mean, etc. of the single levels of satisfaction of a service) may be determined from the determined level of satisfaction of similar users, which may then be used in the recommendation of a service in steps S16 or S28 for use by the particular user from the collected services.

The proposed system is preferably provided for recommending a healthcare service to a particular user (e.g., a patient). Accordingly, a healthcare service is preferably recommended for use by the particular user from the collected healthcare services based on the determined levels of satisfaction and based on the number of times a service was recommended to similar users and/or the number of similar users to which a service was recommended by a care provider. Further, a reputation level of said care providers is preferably taken into account in the recommendation of a healthcare service.

In some embodiments, the proposed system has the following elements as starting point:
A fairly large set U of users (e.g., many thousands) that make use of (part of) a predefined set of services S.
A user profile comprising a list of user profile parameters, e.g., age, gender, nationality, geographical location, medical conditions, medications, etc.
A function profiledist(p,p′) that calculates for each pair of user profiles p and p′ their distance (lower result means that the profiles are closer to each other).
For each user u in U the user profile userprof(u) of that user comprising the user profile parameters pertaining to u.
The subset of services from S used by a specific user u: servused(u).
For each user u in U and each s in servused(u)
   Satisfaction survey(s) from u for s from which a survey satisfaction level surveysatlevel(u,s) can be expressed as a score on a linear scale (higher score for a higher satisfaction from the survey).
   Service usage statistics of s by u, in particular servfreq(u,s) (service frequency, i.e., how often s is used by u) and servduration(u,s) (service duration, i.e., average length of use of s by u).

It shall be noted that a typical scenario for which this holds is a portal/website giving access to multiple services. The portal has drawn a large number of users filling in their own profiles as well as service satisfaction surveys on a regular basis. By monitoring and logging service usage statistics can be generated.

Based on the above-mentioned elements, the recommendation of services can now be refined as follows:

1. Choose a threshold value minclose and determine for each u in U the set simusers(u, minclose) of users similar to u up to a certain threshold (representing the desired minimum level of closeness) by:
u′ ∈simusers(u, minclose)⇔pro filedist(userprof(u), userprof(u))≤minclose.

2. For each u in U define simserv(u, minclose) as the set of all services used by users similar to u:
s ∈ simserv(u, minclose)⇔∃u′ {u′ ∈ simusers(u, minclose)\hat{ }S ∈ servused(u′)}.

3. Define for each u in U and each sin servused(u) an overall satisfaction level satlevel(u,s) as:
survey satlevel(u,s)*servfreq(u,s)*servduration(u,s).

Hereby, many more variants for satlevel can be thought of in addition to this straightforward linear combination of these three contributing factors.

4. Define for each u in U and each s in simserv(u, minclose) a satisfaction level of s for users similar to u, satsimusers(s, u, minclose) as:

average{satlevel(u′,s) u′ ∈ simusers(u, minclose)\hat{ }s ∈ servused(u′)}.

Hereby, variants for satsimusers can be thought of beyond a straightforward average, e.g., giving different weights to different similar users like a higher weight for users closer to u.

Now, to present a recommendation of services to a specific user the results from step 4 are ordered from highest satisfaction level first to lowest satisfaction level last.

The (social) recommendation of services can be thought of by the following simple example. A portal shall be considered providing seniors access to several services, including a shopping assistance service and a haircut at home service. The user profile is an extremely simple one: only age and gender. Profiledist(p, p) is defined as
1−1(abs(age(p)−age(p′))+1) if gender(p)=gender(p) and 1 otherwise.

This yields a value of 0 for same age, same gender; 1 for different gender; between 0 and 1 for same gender, different age.

Now, the following six seniors using this portal (John and Susan are only included for reference later on) shall be considered:

Barbara (75, female), Jennifer (75, female), John (75, male), Linda (74, female), Mary (79, female) and Susan (82, female). Barbara uses neither the shopping assistance service nor the haircut at home service. Jennifer uses only the shopping assistance service. Linda and Mary both use the shopping assistance service and the haircut at home service. For both the shopping assistance service and the haircut at home service a single survey with a linear scale from 1 to 5 has been held and usage statistics (frequency in times per month, duration in average number of minutes) have been observed with the following results:
surveysatlevel(Jennifer, shopping assistance)=3
surveysatlevel(Linda, shopping assistance)=2
surveysatlevel(Mary, shopping assistance)=5
surveysatlevel(Linda, haircut at home)=4
surveysatlevel(Mary, haircut at home)=2
servfreq(Jennifer, shopping assistance)=4
servfreq(Linda, shopping assistance)=2
servfreq(Mary, shopping assistance)=8
servfreq (Linda, haircut at home)=2
servfreq (Mary, haircut at home)=1
servduration(Jennifer, shopping assistance)=60
servduration(Linda, shopping assistance)=45
servduration(Mary, shopping assistance)=90
servduration(Linda, haircut at home)=45
servduration(Mary, haircut at home)=30

For this example, Barbara shall be taken as the target for recommendation and 0.8 as the threshold value minclose. First, the closeness of the profiles of the five others in relation to Barbara shall be calculated:
profiledist(userprof(Barbara), userprof(Jennifer))=0
profiledist(userprof(Barbara), userprof(John))=1
profiledist(userprof(Barbara), userprof(Linda))=0.5
profiledist(userprof(Barbara), userprof(Mary))=0.8
profiledist(userprof(Barbara), userprof(Susan))=0.875.
   From this, it can be found that John and Susan do not qualify as users similar to Barbara: simusers(Barbara, 0.8)={Jennifer, Linda, Mary}.

Since Linda (and Mary) uses both the shopping assistance service and the haircut at home service simserv(Barbara, 0.8)={shopping assistance, haircut at home}.

Next, overall satisfaction levels are calculated:
satlevel(Jennifer, shopping assistance)=3*4*60=720
satlevel (Linda, shopping assistance)=2*2*45=180
satlevel (Mary, shopping assistance)=5*8*90=3600
satlevel (Linda, haircut at home)=4*2*45=360
satlevel (Mary, haircut at home)=2*1*30=60.

This leads to the final computation:
satsimusers(shopping assistance, Barbara, 0.8)=(720+180+3600)/3=1500

and
satsimusers(haircut at home, Barbara, 0.8)=(360+60)/2=210.

In this example the shopping assistance service would merit a much stronger recommendation for Barbara than the haircut at home service. In this specific example the result is dominated by Mary's high levels of survey satisfaction, frequency and duration for the shopping assistance service. In the simple algorithms described above the calculations for different services are not normalized. This gives skewed results that can be improved upon in practical settings. One possibility is to use the deviation (as a percentage) of the overall satisfaction level of a specific user with respect to the average overall satisfaction level of all users of a service instead of the overall satisfaction level of that specific user as an absolute value as used above.

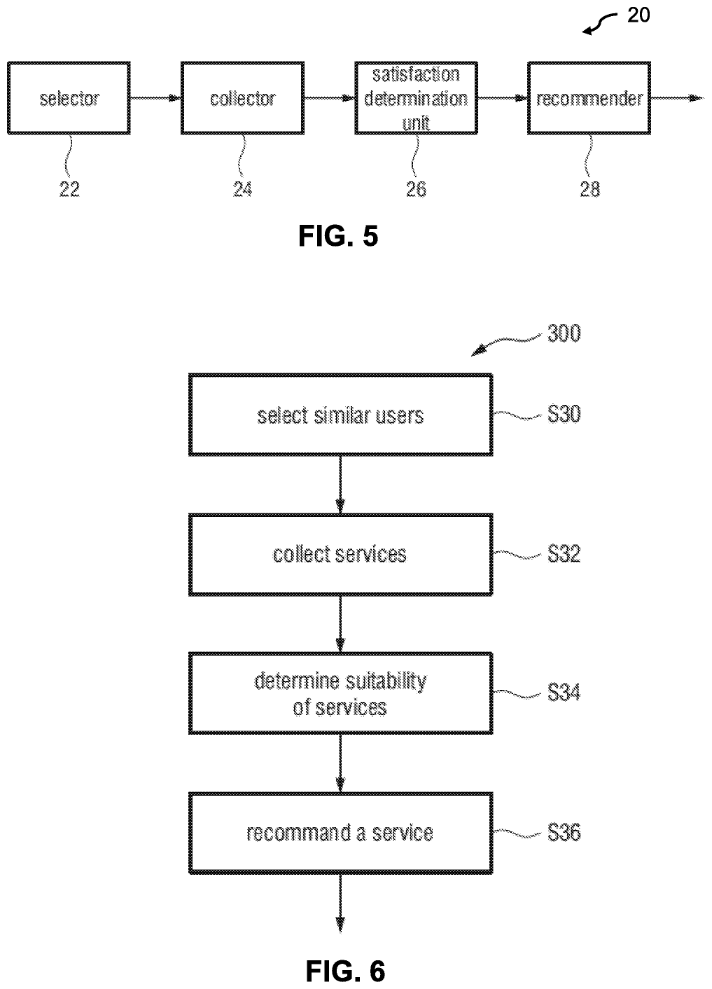

FIG. 5 shows a schematic diagram of a second embodiment of a system 20 for recommending a service to a particular user. The system 20 comprises a selector 22 configured to select from a plurality of users similar users that are similar to the particular user, a collector 24 configured to collect services used by similar users, a level of satisfaction determination unit 26 configured to determine for collected services the level of satisfaction of similar users using the respective collected service, and a recommender 28 configured to recommend a service for use by the particular user from the collected services based on the determined levels of satisfaction. The elements of the system may be implemented as separate or common hardware elements, as software elements or as a combination of hardware and software elements.

According to the above explained embodiments of the proposed system and method the similarity of users, e.g., of a portal or platform, is determined and recommendations for services are based on what services similar users are satisfied with. In the following embodiments of the proposed system and method services are recommended to users, e.g., of a portal or platform, where a multitude of services are available, which is particularly of relevance when the services are healthcare related services and when care providers or other health related professionals such as dieticians or physical therapists are involved in recommending these services to users.

Some embodiments use the user profile to determine the extent to which the user profile matches the intended use of a service. An intended use of a service can include both information for which users this service is appropriate (e.g., a weight loss service is intended for users with a too high Body Mass Index and who are interested in losing weight) as well as information for which users this service is not appropriate (e.g., an extreme sports program is not intended for users with heart problems or osteoporosis). Further embodiments use the input from care providers to determine if a service should be recommended for a particular user. In many cases a care provider will recommend specific services to their patients, as the user profile of these patients is known in the system, this prescribing behavior of care providers can be used to determine which service are endorsed by care providers for which type of users. By combining the prescribing behavior of care providers with a reputation measure of these care providers it is possible to improve the recommendations even further by giving care providers with a high reputation a stronger weight in the calculation of recommendations.

When people can get access to a multitude of services through a single portal or platform, it can be challenging to recommend services that are both relevant and suitable to them. To address this issue it helps to compare the intended use of the services with the user's profile and take into account the recommendations of services by care providers for similar users.

FIG. 6 shows a flow chart of a third embodiment of a method 300, which may be carried out by a system 1 as depicted in FIG. 1. In a first step S30 from a plurality of users, similar users that are similar to the particular user are selected. In a second step S32 services used by similar users are collected. In a third step S34 the suitability of services describing their intended use with respect to a user's profile are determined. In a fourth step S36 a service is recommended for use by the particular user from the collected services based on the suitability of the collected services for the particular user and based on the number of times a service was recommended to similar users and/or the number of similar users to which a service was recommended by a care provider, taking into account a reputation level of said care providers.

Registering the recommendations by care providers for particular patients is fairly straight forward when these care providers make their recommendations through the portal or platform. It assumes that care providers have a way to access the portal or platform and can recommend or prescribe certain services for their patients. Based on the user profiles of these patients this prescribing behavior can be transformed into service recommendations for specific user profiles. This will require a sufficiently large pool of patients to be able to derive a reliable recommendation model. The model can be trained faster by asking the care providers to make their recommendation more explicit by letting them indicate which elements of the user's profile are used as the basis for the recommendation of a specific service.

Determining the reputation of care providers has a wide range of solutions, some of those can be combined to calculate a well-balanced reputation of care provider. Scientific reputation can be based on the generally known H-index, which is an index that attempts to measure both the productivity and impact of the published work of a scientist or scholar. Clinical reputation can be based on the outcomes of the patients of these care providers. This will require adjustments for case mix, as better care providers will most likely also get sicker patients referred to them. Risk adjustment models are available that can help adjust for case mix differences. Patient satisfaction can be based on websites where patients can rate their doctors such as http://www.ratemds.com. A last contributor to reputation could be to let other care providers rate their colleagues.

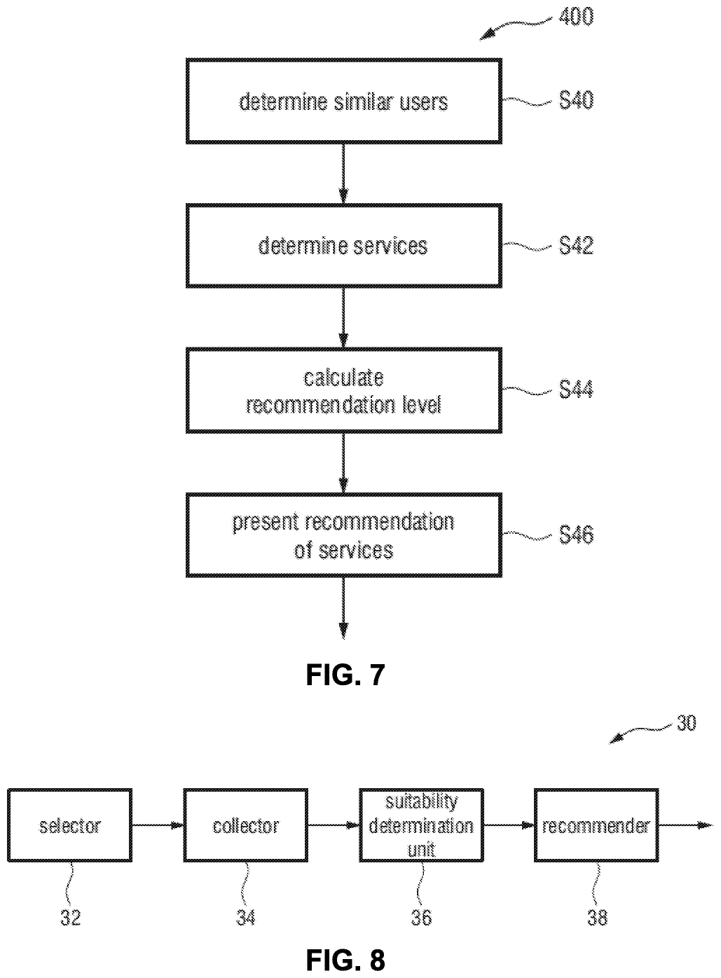

FIG. 7 shows a flow chart of a third embodiment of a method 400, which may be carried out by a system 1 as depicted in FIG. 1. In a first step S40 users similar (e.g., using profiles) to the given user are determined up to a certain threshold (minimum level of closeness). In a second step S42 the services are determined for which a care provider prescribed this service for a similar user that have a minimum level of suitability for the user but for which there is no match between the user's profile and an exclusion criterion in the intended use of this service. In a third step S44 for each service in the collection from step S42, a recommendation level is calculated for this service based on for how many similar users this service was prescribed taking into account the reputation level of the prescribing care provider. This can be adapted by giving different weights to different similar users (higher weight for users closer to the given user). In a fourth step S46 a recommendation of services is presented to the given user by ordering the results from step S44 from highest recommendation level first to lowest recommendation level last.

These recommendations can be extended by taking into account the satisfaction of similar users with these services, as described above. This is particularly of relevance for services for which no care provider recommendations or prescriptions are available.

The service recommendations can be shown directly to the user to find services that could be relevant and suitable for him or to the care provider of the user to help decide which services to prescribe for this user based on what other care providers have recommended for similar users. Suitability of a service for a specific user where services are excluded when the user profile and exclusion criteria match should be interpreted broadly: these may include, for instance, availability (can/does/wants the care provider deliver the service, this may depend on geographic location and local circumstances), affordability (chance of costs being covered by a payer such as insurer, hospital or patient) and accessibility (e.g., available and acceptable transportation facilities for the user).

In the description above, only services that are actually prescribed to similar users are taken into account. In other embodiments, service recommendation scores from care providers are included instead of the binary score of prescribed or not prescribed.

In some embodiments, the proposed system has the following elements as starting point:

A fairly large set U of users (e.g., many thousands) that make use of (part of) a predefined set of services S, and a set P of care providers that prescribe such services to those users.

A user profile comprising a list of user profile parameters (e.g., age, gender, nationality, geographical location, medical conditions, medications, etc.); for each user u in U the user profile userprof(u) of that user comprising the specific user profile parameters pertaining to u.

A function profiledist(up,up′) that calculates for each pair of user profiles up and up′ their distance (lower result means that the profiles are closer to each other)

The subset of care providers from P taking care of a specific user u: careprov(u).

For each user u in U and each p in careprov(u) the set of services from S prescribed by p for u: presserv(p,u).

A set of exclusion criteria E and for each s in S a subset E(s) from E of exclusion criteria related to the intended use of s; and for each user profile up and each subset C from E a function match(up,C) that indicates whether users with profile up should be excluded on basis of one of the criteria in C.

For each user u in U and each s in S a suitability score suitscore(s,u) of s for u.

For each p in P the reputation level of p: replevel(p).

Based on the elements above the recommendation of services can now be refined as follows:

1. Choose a threshold value minclose and determine for each u in U the set simusers(u, minclose) of users similar to u up to a certain threshold (representing the desired minimum level of closeness) by
simusers(u, minclose)={u′|u′ ∈ U\hat{ }profiledist(userprof(u), userprof(u))≤minclose}.

2. Choose a threshold value minsuit and determine for each u in U the set suitserv(u, minsuit) of services suitable for u up to a certain threshold, excluding services considered inappropriate for u by
suitserv(u, minsuit)={s|∃p∃u′ [u′ ∈ simusers(u, minclose)\hat{ }p ∈ careprov(u )\hat{ }s ∈ presserv(p,u′)\hat{ }suitscore(s,u)≥minsuit\hat{ }¬match(userprof(u),E(s))]}

3. Define for each u in U and each s in suitserv(u, minsuit) an overall recommendation level:
reclevel(u,s)=Σ{replevel(p)|∃u′ [u′ ∈ simusers(u, minclose)\hat{ }p ∈ careprov(u′)\hat{ }s∈ presserv(p,u′)]}

Variants for reclevel can be thought of beyond a straightforward sum, e.g., giving different weights to different similar users like a higher weight for users closer to u.

Now, to present a recommendation of services to a specific user the results from step 3 are ordered from highest recommendation level first to lowest recommendation level last.

FIG. 8 shows a schematic diagram of a third embodiment of a system 30 for recommending a service to a particular user. The system 30 comprises a selector 32 configured to select from a plurality of users similar users that are similar to the particular user, a collector 34 configured to collect services used by similar users, a suitability determination unit 36 configured to determine the suitability of services describing their intended use with respect to a user's profile, and a recommender 38 configured to recommend a service for use by the particular user from the collected services based on the suitability of the collected services for the particular user and based on the number of times a service was recommended to similar users and/or the number of similar users to which a service was recommended by a care provider, taking into account a reputation level of said care providers.

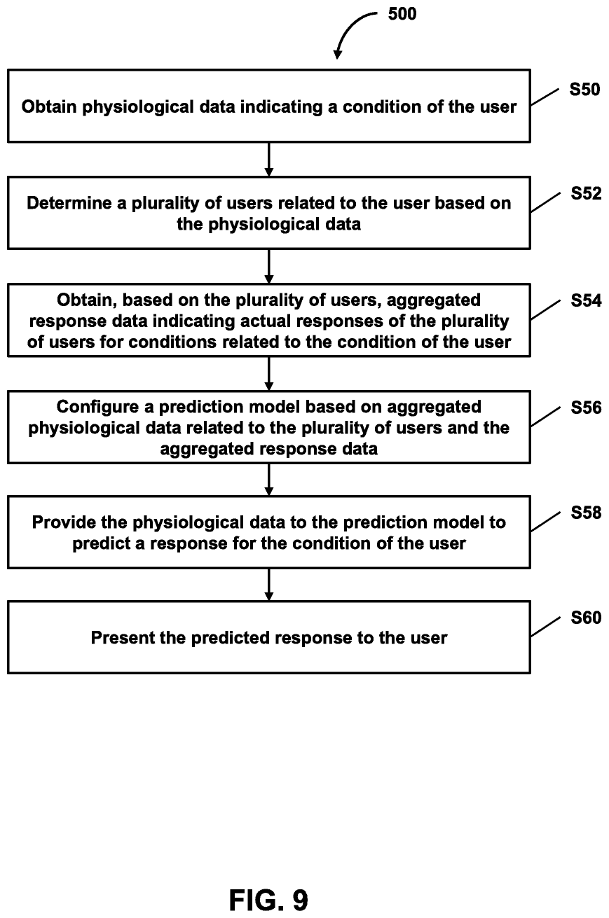

FIG. 9 shows a flow chart for a method 500 of facilitating a response prediction via a prediction model. As an example, the method 500 may be carried out by a system 1 as depicted in FIG. 1. It should be noted that, while one or more operations are described herein with respect to the use of physiological data, those operations may, in some embodiments, be performed using other user profile data (e.g., gender, nationality, geographical location, medications, etc.) or other data.

In step S50, physiological data indicating a condition of the user (or other data related to the user) may be obtained. As an example, the physiological data related to the user may be obtained via a wearable device (e.g., a monitoring device embedded in a body of the user, attached to the user, etc.), via a web interface or mobile application, via one or more databases, etc. The physiological data may include medical conditions of the user, other conditions of the user, or other physiological data related to the user.

In step S52, a plurality of users related to the user may be determined based on the physiological data of the user. As an example, the plurality of users may be determined as being related to the user based on the physiological data of the user. In one use case, for example, a collection of aggregated physiological data of a set of users (e.g., that are in the same age group as the user, that have the same gender as the user, that have the same nationality, that are in the same geographical location, that are on the same medications, etc.) may be obtained. The plurality of user may then be selected from the set of users based on those users' aggregated physiological data being similar to the users' physiological data. In a further use case, the plurality of users may be determined to be users that are similar to the user based on one or more similarity criteria related to condition (e.g., the similar users have the same or similar medical or other condition as the user). Additionally, or alternatively, two users may be determined to be similar based on one or more other similarity criteria (e.g., related to age, gender, nationality, geographical location, medications, or other parameters).

In some embodiments, the physiological data or other data (e.g., other user profile data) related to the user may be provided to a prediction model to predict the plurality of users as users similar to the user. As an example, the prediction model may include a neural network or other machine learning model trained to predict one or more classifications of the user, and such classifications of the user may be used to determine whether or the extent to which other users are similar to the user (e.g., based on the extent to which the classifications of the others match the classifications of the user). As a further example, the prediction model may use the predicted classifications to query a database (e.g., training data database(s) 5 of prediction database(s) 4 or other databases) of users to obtain results indicating the plurality of users as being users similar to the user.

In step S54, based on the plurality of users, aggregated response data indicating actual responses of the plurality of users (e.g., for conditions related to the condition of the user) may be obtained. As an example, the aggregated response data may indicate the actual responses of the plurality of users after those users were presented with one or more responses recommended to be taken for the respective conditions of those users (or were otherwise actually performed by the plurality of users). In one use case, the actual responses of the plurality of users may include actual usage of one or more devices or services by the plurality of users (e.g., usage frequency, usage duration, or other usage data).

In step S56, a prediction model may be configured based on the aggregated physiological data and the aggregated response data related to the plurality of users. As an example, the aggregated physiological data may be provided to the prediction model, and the prediction model may predict responses for the related conditions. The prediction model may then be updated based on the predicted responses and the aggregated response data related to the plurality of users (e.g., data indicating the actual responses of the users). As an example, the aggregated response data related to the plurality of users may be provided to the prediction model as reference feedback, and the prediction model may update one or more portions of the prediction model based on the aggregated response data and the predicted responses. As indicated herein, in some embodiments, the predicted responses may include recommended use of one or more devices or services for the other users, and the actual responses of the plurality of users may include actual usage of the devices or services by the plurality of users (e.g., usage frequency, usage duration, or other usage data).

In step S58, subsequent to the configuration of the prediction model, the physiological data of the user may be provided to the prediction model to predict a response for the condition of the user. As an example, the predicted response for the user may include recommended use of a device or service for the condition of the user. In step S60, the predicted response may be presented to the user (e.g., via a user interface of a wearable device or other client device).

In some embodiments, response data related to the user may be obtained, and the prediction model may be updated based on the response data. As an example, the response data may indicate an actual response of the user for the condition (e.g., the user's actual response after being presented with the predicted response). The response data may be provided to the prediction model as reference feedback, and the prediction model may assess the actual response against the predicted response and update one or more configurations of the prediction model based on the assessment. In this way, for example, the prediction model may be configured to provide more accurate predictions personalized for the user.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium can be, for example, without limitation, an electronic, magnetic, optical, electromagnetic, infrared, or semiconductor system, or a propagation medium. Non-limiting examples of a computer readable medium include a semiconductor or solid-state memory, magnetic tape, a removable computer diskette, a random access memory (RAM), a read-only memory (ROM), a rigid magnetic disk, and an optical disk. Optical disks may include compact disk read only memory (CD-ROM), compact disk read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

Although example embodiments have been described in detail for the purpose of illustration, it is to be understood that such detail is solely for that purpose and that embodiments are not limited to the disclosed embodiments, but, on the contrary, are intended to cover modifications and equivalent arrangements that are within the scope of the appended claims. For example, it is to be understood that embodiments contemplate that, to the extent possible, one or more features of any embodiment can be combined with one or more features of any other embodiment.

As used throughout this application, the word “may” is used in a permissive sense (i.e., meaning having the potential to), rather than the mandatory sense (i.e., meaning must). The words “comprise,” “comprising,” “comprises,” “include”, “including”, and “includes” and the like mean including, but not limited to. As used throughout this application, the singular forms “a,” “an,” and “the” include plural referents unless the context clearly indicates otherwise, and notwithstanding the use of other terms and phrases for one or more elements, such as “one or more.” The term “or” is non-exclusive (i.e., encompassing both “and” and “or”), unless the context clearly indicates otherwise. Further, unless otherwise indicated, statements that one value or action is “based on” another condition or value encompass both instances in which the condition or value is the sole factor and instances in which the condition or value is one factor among a plurality of factors. Unless the context clearly indicates otherwise, statements that “each” instance of some collection have some property should not be read to exclude cases where some otherwise identical or similar members of a larger collection do not have the property, i.e., each does not necessarily mean each and every.

Additional example embodiments are provided with reference to the following enumerated embodiments:

A1. A method comprising: selecting from a plurality of users similar users that are similar to the particular user, collecting services used by similar users, determining for collected services the level of satisfaction of similar users using the respective collected service, and recommending a service for use by the particular user from the collected services based on the determined levels of satisfaction of similar users
A2. The method of embodiment A1, further comprising: determining the level of similarity for a plurality of users; and selecting similar users based on the determined level of similarity.
A3. The method of any of embodiments A1-A2, wherein the instructions further cause the processor to determine for collected services the level of satisfaction of similar users based on direct feedback from said similar users and/or actual usage of services by said similar users.
A4. The method of embodiment A3, further comprising an interface for obtaining direct feedback and/or information about actual usage of services by users.
A5 The method of embodiment A3, further comprising measurement means for measuring actual usage of services by users including usage frequency and/or usage duration.
A6. The method of any of embodiments A1-A5, further comprising: determining the level of similarity for a plurality of users based on the profile of the users; selecting similar users based on the determined level of similarity; determining the suitability of collected services describing their intended use with respect to a user's profile; and recommending a service for use by the particular user from the collected services based on the determined levels of satisfaction and the suitability of the collected services for the particular user.
A7. The method of any of embodiments A1-A6, further comprising: recommending a service for use by the particular user from the collected services based on the determined levels of satisfaction and based on the number of times a service was recommended to similar users and/or the number of similar users to which a service was recommended.
A8. The method of any of embodiments A1-A7, further comprising: recommending a healthcare service for use by the particular user from the collected healthcare services based on the determined levels of satisfaction and based on the number of times a service was recommended to similar users and/or the number of similar users to which a service was recommended by a care provider.
A9. The method of any of embodiments A1-A8, further comprising: taking into account a reputation level of said care providers in the recommendation of a healthcare service.
A10. The method of any of embodiments A1-A9, further comprising: determining for the respective collected services a combined satisfaction level from the determined level of satisfaction of similar users and to recommend a service for use by the particular user from the collected services based on the combined satisfaction level.
A11. A method comprising: selecting from a plurality of users similar users that are similar to the particular user; collecting services used by similar users; determining the suitability of services describing their intended use with respect to a user's profile; and recommending a service for use by the particular user from the collected services based on the suitability of the collected services for the particular user and based on the number of times a service was recommended to similar users and/or the number of similar users to which a service was recommended by a recommendation giver taking into account a reputation level of said recommendation givers.
A12. The method of embodiment A11, further comprising: determining the suitability of a service for a user based on the profile of the user and the intended use of the service.
A13. The method of embodiment A12, using one or more exclusion criteria for determining the suitability of a service for a user, an exclusion criterion indicating which users can or shall not use a particular service.
A14. The method of embodiment A13, further comprising: using one or more exclusion criteria that take into account availability, affordability and/or accessibility of a service for a user.
A15. The method of any of embodiments A11-A14, further comprising: determining the reputation level of said recommendation givers.
B1. A method comprising: obtaining physiological data related to the user, the physiological data indicating a condition of the user; determining a plurality of users related to the user based on the physiological data; obtaining based on the plurality of users, aggregated response data indicating actual responses of the plurality of users to presentation of predicted responses for conditions related to the condition of the user; configuring a prediction model based on aggregated physiological data related to the plurality of users, the aggregated response data, or other data; and subsequent to the configuration of the prediction model, providing the physiological data to the prediction model to predict a response for the condition of the user; and causing the predicted response to be provided for presentation to the user.
B2. The method of embodiment B1, wherein configuring the prediction model comprises: providing the aggregated physiological data related to the plurality of users to the prediction model, the prediction model predicting responses for the related conditions based on the aggregated physiological data, the aggregated physiological data indicating the related conditions; and providing the aggregated response data related to the plurality of users to the prediction model as reference feedback for the prediction model's prediction of the responses to update the prediction model.
B3. The method of any of embodiments B1-B2, further comprising: obtaining response data related to the user, the response data indicating an actual response of the user to presentation of the predicted response to the user; and updating the prediction model based on the physiological data and the response data.
B4. The method of embodiment B3, wherein updating the prediction model comprises providing the response data related to the user to the prediction model as reference feedback for the prediction model's prediction of the response to update the prediction model.
B5. The method of any of embodiments B1-B4, wherein determining the plurality of users comprises providing the physiological data related to the user to a second prediction model to predict the plurality of users as being related to the user.
B6. The method of any of embodiments B1-B5, wherein determining the plurality of users comprises determining the plurality of users as being similar to the user based on the physiological data.
B7. The method of any of embodiments B1-B6, wherein the predicted responses comprise recommended use of one or more devices or services for the plurality of users, and the actual responses of the plurality of users comprises actual usage of the one or more devices or services by the plurality of users.
B8. The method of embodiment B7, wherein the actual usage of the one or more devices of services comprises usage frequency or usage duration of the one or more devices or services, wherein the aggregated response data indicates the usage frequency or the usage duration of the one or more devices or services.
B9. The method of any of embodiments B1-B8, wherein the predicted response for the condition of the user comprises recommended use of a device or service for the user.
B10. The method of any of embodiments B1-B9, wherein the prediction model comprises a neural network.
B11. The method of embodiment B10, wherein configuring the prediction model comprises providing the aggregated physiological data related to the plurality of users to the neural network for the neural network to predict responses for the plurality of users (e.g., for conditions related to the condition of the user); and providing the aggregated response data related to the plurality of users to the neural network as reference feedback for the neural network's prediction of the responses, the neural network configuring one or more layers of the neural network based the aggregated response data and the predicted responses.
C1. One or more tangible, non-transitory, machine-readable media storing instructions that, when executed by one or more processors, effectuation operations comprising those of any of embodiments A1-A15 and B1-B11.
C2. A system comprising: one or more processors; and memory storing computer program instructions that, when executed by the one or more processors, cause the one or more processors to effectuate operations comprising those of any of embodiments A1-A15 and B1-B11.

1. A system for facilitating response predictions for a condition, the system comprising:
a computer system that comprises one or more processors executing computer program instructions that, when executed, cause the computer system to:
obtain, via a wearable device attached to a user, physiological data related to the user, the physiological data indicating a condition of the user;
determine a plurality of users related to the user based on the physiological data;
obtain, based on the plurality of users, combined response data indicating actual responses of the plurality of users to presentation of predicted responses for conditions related to the condition of the user;
provide combined physiological data related to the plurality of users to a neural network for the neural network to predict responses for the related conditions, the combined physiological data indicating the related conditions;
provide the combined response data related to the plurality of users to the neural network as reference feedback for the neural network's prediction of the responses, the neural network configuring one or more layers of the neural network based the combined response data and the predicted responses; and
subsequent to the configuration of the neural network, provide the physiological data related to the user to the neural network for the neural network to predict a response for the condition of the user; and
cause, via a user interface in communication with the wearable device, presentation of the predicted response to the user.

2. The system of claim 1, wherein the computer system is caused to:
provide response data related to the user to the neural network as reference feedback for the neural network's prediction of the response, the response data indicating an actual response of the user to presentation of the predicted response to the user, the neural network configuring one or more layers of the neural network based on the response data and the predicted response.

3. The system of claim 1, wherein determining the plurality of users comprises providing the physiological data related to the user to a second neural network for the second neural network to the plurality of users as being related to the user based on the physiological data related to the user.

4. A method comprising:
obtaining, by one or more processors, physiological data related to the user, the physiological data indicating a condition of the user;
determining, by one or more processors, a plurality of users related to the user based on the physiological data;
obtaining, by one or more processors, based on the plurality of users, combined response data indicating actual responses of the plurality of users to presentation of predicted responses for conditions related to the condition of the user;
configuring, by one or more processors, a prediction model based on combined physiological data related to the plurality of users and the combined response data; and
subsequent to the configuration of the prediction model, providing, by one or more processors, the physiological data to the prediction model to predict a response for the condition of the user; and
causing, by one or more processors, the predicted response to be provided for presentation to the user.

5. The method of claim 4, wherein configuring the prediction model comprises:
providing the combined physiological data related to the plurality of users to the prediction model, the prediction model predicting responses for the related conditions based on the combined physiological data, the combined physiological data indicating the related conditions; and
providing the combined response data related to the plurality of users to the prediction model as reference feedback for the prediction model's prediction of the responses to update the prediction model.

6. The method of claim 4, further comprising:
obtaining response data related to the user, the response data indicating an actual response of the user to presentation of the predicted response to the user; and
updating the prediction model based on the physiological data and the response data.

7. The method of claim 6, wherein updating the prediction model comprises providing the response data related to the user to the prediction model as reference feedback for the prediction model's prediction of the response to update the prediction model.

8. The method of claim 4, wherein determining the plurality of users comprises providing the physiological data related to the user to a second prediction model to predict the plurality of users as being related to the user.

9. The method of claim 4, wherein determining the plurality of users comprises determining the plurality of users as being similar to the user based on the physiological data.

10. The method of claim 4, wherein the predicted responses comprise recommended use of one or more devices or services for the plurality of users, and the actual responses of the plurality of users comprises actual usage of the one or more devices or services by the plurality of users.

11. The method of claim 10, wherein the actual usage of the one or more devices of services comprises usage frequency or usage duration of the one or more devices or services, wherein the combined response data indicates the usage frequency or the usage duration of the one or more devices or services.

12. The method of claim 4, wherein the predicted response for the condition of the user comprises recommended use of a device or service for the user.

13. The method of claim 4, wherein the prediction model comprises a neural network.

14. One or more non-transitory computer-readable media comprising one or more instructions that, when executed by one or more processors, cause operations comprising:
obtaining user profile data related to the user, the user profile data indicating a condition of the user;
determining a plurality of users related to the user based on the user profile data;
obtaining combined response data indicating actual responses of the plurality of users for conditions related to the condition of the user;
generating a response for the condition of the user based on (i) combined physiological data related to the users and (ii) the combined response data indicating the actual responses of the plurality of users; and
causing the response to be provided for presentation to the user.

15. The non-transitory computer-readable media of claim 14, the instructions further comprising:
configuring a prediction model based on the combined physiological data related to the plurality of users and the combined response data indicating the actual responses of the plurality of users; and
subsequent to the configuration of the prediction model, providing the physiological data to the prediction model to generate the response for the condition of the user.

16. The non-transitory computer-readable media of claim 15, wherein the prediction model comprises a neural network.

17. The non-transitory computer-readable media of claim 14, wherein determining the plurality of users comprises determining the plurality of users as being similar to the user based on the user profile data.

18. The non-transitory computer-readable media of claim 14, wherein the predicted responses comprise recommended use of one or more devices or services for the plurality of users, and the actual responses of the plurality of users comprises actual usage of the one or more devices or services by the plurality of users.

19. The non-transitory computer-readable media of claim 18, wherein the actual usage of the one or more devices of services comprises usage frequency or usage duration of the one or more devices or services, wherein the combined response data indicates the usage frequency or the usage duration of the one or more devices or services.

20. The non-transitory computer-readable media of claim 14, wherein the generated response for the condition of the user comprises recommended use of a device or service for the user.
